(19) United States
(12) Patent Application Publication
(10) Pub. No.: US 2023/0355500 A1
(43) Pub. Date: Nov. 9, 2023

(54) TOPICAL COMPOSITION

(71) Applicant: LILIGER LTD, Herzliya (IL)

(72) Inventors: Eyal S. RON, Lexington, MA (US); Dov TAMARKIN, Tel Aviv (IL); Avner SHEMER, Netanya (IL)

(21) Appl. No.: 18/221,529

(22) Filed: Jul. 13, 2023

Related U.S. Application Data
(63) Continuation-in-part of application No. PCT/IL2022/050056, filed on Jan. 13, 2022.
(60) Provisional application No. 63/138,702, filed on Jan. 18, 2021.
(60) Provisional application No. 63/137,732, filed on Jan. 15, 2021.

(51) Int. Cl. A61K 8/67; A61K 8/44; A61K 8/66; A61K 8/64; A61Q 19/00; A61Q 19/08

(52) U.S. Cl. CPC A61K 8/675; A61K 8/44; A61K 8/66; A61K 8/64; A61Q 19/007; A61Q 19/08; A61K 2800/88

(57) ABSTRACT

A topical composition for application to the skin, comprising an NO-donor and a counter-NO agent, which balances the effect of the NO-donor in the skin. The present invention relates to novel topical compositions designed for application to the skin. The composition comprises an NO-donor, which serves as a source of nitric oxide, and a counter-NO agent that acts to balance the effects of the NO-donor on the skin. The combined use of the NO-donor and the counter-NO agent in the composition provides a unique and beneficial therapeutic effect.

### CROSS REFERENCE TO RELATED APPLICATIONS

This application is a Continuation-in-part of PCT Patent Application No. PCT/IL2022/050056 having International filing date of Jan. 13, 2022, which claims the benefit of priority of U.S. Provisional Patent Application Nos. 63/137,732, filed Jan. 15, 2021 and 63/138,702, filed Jan. 18, 2021, the contents of which are all incorporated herein by reference in their entirety.

### FIELD OF THE INVENTION

The invention relates to topical compositions comprising a first agent, which is a nitric oxide donor and a second agent, having the property of suppressing the effect of NO; and methods of topical treatment using such compositions.

### BACKGROUND OF THE INVENTION

Nitric oxide (“NO”) is a small gaseous molecule also known as nitrogen monoxide with the chemical formula NO, which has been shown to be a very important signaling molecule in living organisms including the human body, and particularly the skin and its underlying tissues.

In the skin, NO enhances cutaneous microcirculation, it is involved in skin pigmentation through ultraviolet induced melanogenesis, it has been reported to promote wound healing by cellular proliferation and angiogenesis and to have antimicrobial properties against micro-organisms. NO also plays an important role in T-cell mediated diseases of the skin, and it has both pro and anti-apoptotic properties depending on its concentration, cell type, and availability of other substrates.

Thus, it would be beneficial to administer NO into the skin; however, it is impractical to apply NO in its gas state to the skin, in a way that will facilitate such biological effects or impart a therapeutic effect, and thus, the use of certain NO-donors has been proposed.

While NO possesses beneficial effects, as summarized above, it also has the potential to impart notable cutaneous side effects, due to its potent pro-inflammatory properties. NO is known to mediate cutaneous oedema and inflammation and may contribute to impaired skin barrier function.

Thus, there remains an unmet need for administering effective amounts of NO into the skin, while suppressing its untoward pro-inflammatory properties.

### SUMMARY OF THE INVENTION

An aspect of the invention pertains to a topical composition for application to the skin, comprising an NO-donor and a counter-NO agent, which balances the effect of the NO-donor in the skin.

In one or more embodiments, the NO-donor is selected from the group consisting of an inorganic nitrite, an inorganic nitrate, an organic nitrite, an organic nitrate, sodium nitroprusside, molsidomine, diazeniumdiolates, S-nitrosothiols, mesoionic oxatriazole, iron-sulphur nitrosyls, Sinitrodil, L-arginine and L-citrulline; and cosmetically or pharmaceutically acceptable salts, isomers, analogs and derivatives thereof.

In one or more embodiments, the NO-donor is selected from the group consisting of an inorganic nitrite, a nitrate salt, an organic nitrite, an organic nitrate, an ester of nitric acid, sodium nitroprusside, molsidomine, diazeniumdiolates, S-nitrosothiols, mesoionic oxatriazole, an iron-sulphur nitrosyl, a nitrite ester of a sugar, a nitrate ester of a sugar, a nitrite ester of a polyol, a nitrate ester of a polyol, arginine, citrulline, nitroglycerin, ethylene glycol dinitrate, isopropyl nitrate, amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, butane-1,2,4-triol-trinitrate, erythrityl tetranitrate, pentaerythrityl tetranitrate, sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate, propatylnitrate, nicorandil, apresoline, diazoxide, hydralazine, hydrochlorothiazide, minoxidil, pentaerythritol, tolazoline; and cosmetically or pharmaceutically acceptable salts, isomers, analogs and derivatives thereof.

In one or more embodiments, the NO-donor is nitroglycerine. In one or more embodiments, the concentration of nitroglycerine is selected from the group consisting of between about 4% and about 12%, between about 4% and about 8%, and between about 8% and about 12%.

In one or more embodiments, the NO-donor is L-arginine, a salt, an isomer, an analog, or a derivative thereof.

In one or more embodiments, the concentration of arginine is selected from the group consisting of from about 6% to about 25%, about 6% to about 12%, about 6% to about 8%, from about 8% to about 10%, from about 10% to about 14%, from about 14% to about 18%, and from about 18% to about 25%.

In one or more embodiments, the NO-donor is L-citrulline, a salt, an isomer, an analog, or a derivative thereof.

In one or more embodiments, the concentration of citrulline is selected from the group consisting of from about 4% to about 20%, from about 4% to about 6%, from about 6% to about 10%, from about 10% to about 14% and from about 14% to about 20%.

In one or more embodiments, the counter-NO agent is selected from the group consisting of a NOS inhibitor, a neuronal NOS (nNOS) inhibitor, an inducible NOS (iNOS) inhibitor, an endothelial NOS (eNOS) inhibitor, a suppressor of the expression of eNOS by cells, an inhibitor of NOS production, and a combination thereof.

In one or more embodiments, the counter-NO agent is selected from the group consisting of 1-NG-nitroarginine, NG-Nitro- L-Arginine Methyl Ester, NG-monomethyl-l-arginine, the methyl ester of NG-nitro-L-arginine, L-NG-Nitroarginine, NG-amino-L-arginine, NG, NG-dimethyl-arginine, S-Ethylisothiouronium diethylphosphate, L-Thiocitrulline, S-Methyl-L-Thiocitrulline, Agmatine, 7-Bromonitroindazole, 1-[2-(Trifluoromethyl)phenyl-imidazole, S-(2-Aminoethyl) isothiourea, Methylene blue, 1H41,2,4]Oxadiazole[4,3-a]quinoxalin-1-one, and a combination thereof.

In one or more embodiments, the counter-NO agent is nicotinamide, a salt, an isomer, an analog or a derivative thereof.

In one or more embodiments, the concentration of nicotinamide is from about 0.25% to about 10%, about 0.25% to about 4%, 1% to about 2%, from about 2% to about 4%, from about 4% to about 6%, from about 6% to about 8%, or from about 8% to about 10%.

In one or more embodiments, the ratio between the NO-donor and the counter-NO agent in the topical composition is between about 3:5 to about 100:1, about 2.5:1 to about 40:1, about 1:2 and about 8:1, between about 1:2 and about 1:1, between about 1:1 and about 2:1, between about 2:1 and about 4:1, or between about 4:1 and about 8:1.

In one or more embodiments, when the NO-donor is arginine and the counter-NO agent is nicotinamide the ratio between the arginine and nicotinamide in the topical composition is between about 2.5:1 to about 40:1, about 3:5 to about 100:1, about 2.5:1 to about 5:1, about 2:1 to about 8:1, between about 2:1 and about 4:1, between about 4:1 and about 6:1, or between about 6:1 and about 8:1.

In one or more embodiments, when the NO-donor is citrulline and the counter-NO agent is nicotinamide, the ratio between the citrulline and nicotinamide in the topical composition is between about 2:1 and about 8:1, between about 2:1 and about 4:1, between about 4:1 and about 6:1, or between about 6:1 and about 8:1.

In one or more embodiments, upon topical application to the skin, the composition exerts a tolerable tingling effect.

Yet another aspect pertains to a kit comprising a first topical composition for application to the skin according to the herein disclosure, and a second composition comprising at least one neurotoxin.

In one or more embodiments, the at least one neurotoxin is selected from the group consisting of acetyl hexapeptide-3, beta-Ala-Pro-Dab-NH-benzyl×2AcOH, acetyl hexapeptide-1, Lys-Thr-Thr-Lys-Ser, botulism toxin, an inhibitor of SNARE complex formation and catecholamine release, an antagonism of muscular nicotinic acetylcholine receptor, an inhibitor of the release of the acetylcholine (ACh) neurotransmitter into the synapse, the peptide AC-gly glu-met-gln-arg-arg-NH2, Tripeptide-3, an antagonist at the acetylcholine postsynaptic membrane receptor, Pentapeptide-3, and derivatives, analogs, or salts thereof.

In one or more embodiments, the second composition comprises about 5% acetyl hexapeptide-3, about 2% beta-Ala-Pro-Dab-NH-benzyl×2 AcOH, and about 3% acetyl hexapeptide-1.

In one or more embodiments, the first composition or the second composition further comprise one or more inhibitors of hyaluronidase.

Another aspect of the invention pertains to a method of treating, alleviating, or preventing a skin condition, comprising administering topically to the skin an effective amount of a composition as herein disclosed.

Yet another aspect of the invention pertains to a composition as herein disclosed for use in treating, alleviating, or preventing a skin condition, comprising administering topically to the skin of a subject an effective amount of a composition as herein disclosed.

In one or more embodiments, the skin condition is associated with skin ageing. In one or more embodiments, the skin condition includes at least one of the following: light-induced skin ageing, increased trans-epidermal water loss, skin dryness, fine lines, wrinkles, hyperpigmentation, and skin discoloration.

In one or more embodiments, the composition is a first composition and wherein the method further comprises applying to the skin a second composition comprising at least one neurotoxin, and wherein the application to the skin of the first composition is conducted in concurrence with, prior to, or following the administration of the second composition.

In one or more embodiments, the application of the composition augments the effect of the neurotoxin and/or prolongs the duration of the effect of the neurotoxin, and/or alleviates the inflammation and or the atrophy that is induced following administration of the neurotoxin.

In one or more embodiments, the first composition or the second composition further comprise one or more inhibitors of hyaluronidase.

In one or more embodiments, the neurotoxin is selected from the group consisting of botulism toxin, an inhibitor of SNARE complex formation and catecholamine release, an antagonism of muscular nicotinic acetylcholine receptor, an inhibitor of the release of the acetylcholine (ACh) neurotransmitter into the synapse, acetyl hexapeptide-3, the peptide AC-gly glu-met-gln-arg-arg-NH2, Tripeptide-3, beta-Ala-Pro-Dab-NH-benzyl×2AcOH, acetyl hexapeptide-1, an antagonist at the acetylcholine postsynaptic membrane receptor, Pentapeptide-3, Lys-Thr-Thr-Lys-Ser; and derivatives, analogs or salts thereof.

In one or more embodiments, the NO-donor is arginine, and the counter-NO agent is nicotinamide, wherein the application is conducted in concurrence with the dermal administration of a neurotoxin. In the context of the present invention, dermal administration means the delivery of a substance, where the intended site of application is the skin. It involves applying such substance directly onto the skin, which may result in the delivery of active agents into the skin, sub-dermally or trans-dermally.

In one or more embodiments, the concentration of arginine in the composition is selected from the group consisting of from about 6% to about 25%, from about 6% to about 8%, from about 8% to about 10%, from about 10% to about 14%, from about 14% to about 18%, and from about 18% to about 25%.

In one or more embodiments, the concentration of nicotinamide in the composition is from about 1% to about 2%, from about 2% to about 4%, from about 4% to about 6%, from about 6% to about 8%, or from about 8% to about 10%.

In one or more embodiments, the ratio between the arginine and nicotinamide in the topical composition is between about 2:1 and about 8:1, between about 2:1 and about 4:1, between about 4:1 and about 6:1, or between about 6:1 and about 8:1.

In one or more embodiments, the NO-donor is citrulline and the counter-NO agent is nicotinamide, wherein the application is conducted in concurrence with the dermal administration of a neurotoxin.

In one or more embodiments, the concentration of citrulline in the composition is selected from the group consisting of from about 4% to about 20%, from about 4% to about 6%, from about 6% to about 10%, from about 10% to about 14%, and from about 14% to about 20%, and the concentration of nicotinamide is selected from the group consisting of from about 1% to about 2%, from about 2% to about 4%, from about 4% to about 6%, from about 6% to about 8%, or from about 8% to about 10%.

In one or more embodiments, the application of the composition increases the cutaneous microcirculation and the supplies of oxygen and nutrients to the skin tissues.

In one or more embodiments, the application of the composition augments the effect of the neurotoxin and/or prolongs the duration of the effect of the neurotoxin.

In one or more embodiments, the application of the composition is conducted prior to administration of the neurotoxin, or on a continuous basis following the administration of the neurotoxin.

In one or more embodiments, the application of the composition alleviates the inflammation and/or the atrophy that is induced following administration of the neurotoxin. Neurotoxin injections are administered repeatedly over extended periods or in high doses, and it is therefore possible for the atrophy to affect not only the targeted muscles but also nearby structures, such as surrounding soft tissues or fat deposits. This can result in a localized loss of volume or a sunken appearance in the treated area.

In one or more embodiments, the composition is in a form selected from the group consisting of a liquid, a solution, an emulsion, a lotion, a cream, a gel, a foam, a lipstick, a mask, and a serum.

### DETAILED DESCRIPTION OF THE INVENTION

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It should be noted that the terminology utilized herein is solely intended to describe specific embodiments and should not be construed as limiting in any way. Unless defined otherwise, all technical and scientific terms used herein have the same meaning as commonly understood by one of ordinary skill in the art to which this disclosure belongs.

The invention relates to a topical composition comprising an NO-donor, together with a counter-NO agent, which balances the effect of the NO in the skin.

The invention further relates to a method of treating, alleviating, or preventing a dermatological, cosmetic, or mucosal condition, comprising administering topically to a subject having said disorder a therapeutically effective amount of any of the compositions described herein.

### Nitric Oxide Donors

Nitric oxide, having the molecular formula NO, is a very small molecule that, upon release from a composition, can migrate fast and reach its target site. Nitric oxide is a remarkably versatile biological messenger. The chemical properties of NO are crucial in defining its biological roles, both as a transcellular signal in the cardiovascular and nervous systems and as a cytotoxic antipathogenic agent released during an inflammatory response. Among other properties, NO is known to be an antimicrobial. The term “antimicrobial” as used herein includes, but is not limited to, a destructive or inhibitory effect on the growth of bacteria, fungi, viruses, and other microbial and sub-microbial pathogens; or the capacity to inhibit the growth of or to destroy bacteria, fungi, viruses, and other microbial and sub-microbial pathogens. NO can further have anti-inflammatory effects and skin revitalizing effects and it is also known to enhance wound healing. It can further be used to treat various dermatoses and keratoses.

Upon penetration into and through the skin, NO can cause peripheral vasodilation, otherwise described as enhanced blood flow in the skin, which facilitates improved provision of oxygen and nutrients into the subcutaneous tissues, the dermis, and the epidermis.

Peripheral vasodilation is also known to lower systolic and diastolic blood pressure.

Various compounds are known to be NO-donors. In an exemplary embodiment, the NO-donor can be selected from several classes, including, but not limited to inorganic nitrite and nitrate salts (e.g., sodium nitrite and sodium nitrate), organic nitrites and nitrates, sodium nitroprusside, molsidomine and its metabolites, diazeniumdiolates, S-nitrosothiols, mesoionic oxatriazole and derivatives thereof, iron-sulphur nitrosyls, Sinitrodil and derivatives thereof and the amino acids L-arginine and L- citrulline. For clarification purposes, nitrite NO₂⁻ and nitrate is NO₃⁻.

In an embodiment of the present invention, the organic NO-donor includes at least one organic nitrite, which includes esters of nitric acid and may be an acyclic or cyclic compound. For instance, the organic nitrate may be nitroglycerin, ethylene glycol dinitrate; isopropyl nitrate; amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, butane-1,2,4-triol-trinitrate; erythrityl tetranitrate; pentaerythrityl tetranitrate; sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatylnitrate, nitrite esters of sugars, nitrate esters of sugars, nitrite esters of polyols, nitrate esters of polyols, nicorandil, apresoline, diazoxide, hydralazine, hydrochlorothiazide, minoxidil, pentaerythritol, tolazoline, scoparone (6,7-dimethoxycoumarin) and cosmetically or pharmaceutically acceptable salts, isomers, analogs and derivatives thereof.

By way of example, the following equations show the equilibrium reaction of a nitrite salt to form nitric oxide:

A. MN0₂+HA [symbol] HN0₂

B. 2 HNO2 [symbol] N₂0₃+H₂O H₂O+NO+NO₂

C. 3 HN0₂ [symbol] 2 NO+NO₃⁻+H₂0+H+

In this case, the NO-donor is a nitrite salt (MN0₂), such as sodium nitrite.

In an embodiment, arginine is the NO-donor, and NO is produced from arginine in the skin in a reaction that is catalyzed by the enzyme nitric oxide synthase, as illustrated below:

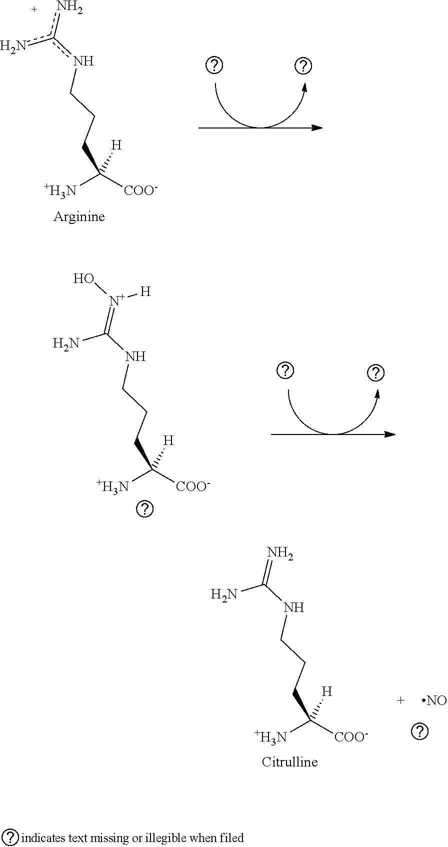

According to U.S. Pat. No. 5,895,658, topical administration of a 12.5% arginine preparation helps in the induction of hair growth, however, when applied to cool skin, elevation of skin temperature, up to 10° C. was noticed, which may cause inconvenience to the treated person and therefore, incompliance to treatment.

L-Arginine and L-citrulline can be used as a free base or as a salt, such as a hydrochloride salt; or any salts, isomers, analogs, and derivatives, that converts into arginine through a chemical or an enzymatic process.

In an embodiment of the present invention, the NO-donor is arginine (arginine, 1-arginine and L-arginine are used herein interchangeably); and in another embodiment, the NO-donor is citrulline (citrulline, 1-citrulline and L-citrulline are used herein interchangeably).

In an embodiment, the NO-donor is linked to a polymer. In a further embodiment the NO-donor is N-diazeniumdiolate. In yet a further embodiment, the NO-donor comprises N-diazeniumdiolate

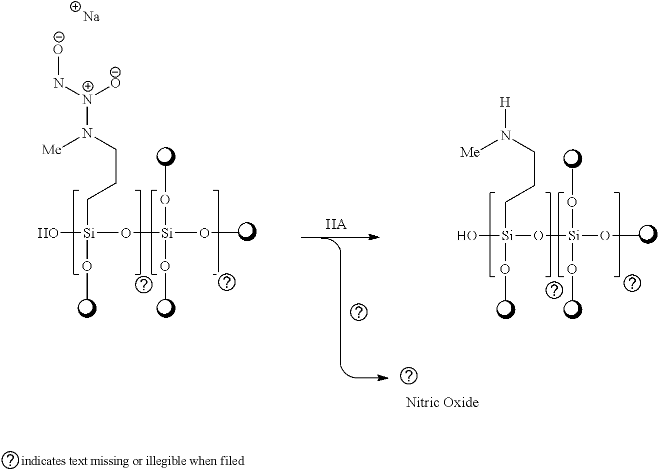

bound to a polymer; and in an additional embodiment, the NO-donor comprises a polysiloxane polymer backbone that contains covalently bound N-diazeniumdiolate nitric oxide donors throughout the polymeric structure, as illustrated in the following scheme.

In this case, the NO-donor is bound to a polymer and it releases NO gradually upon application to the skin, as detailed above. In this case, the NO-donor is a nitrite salt (MN0₂, wherein M is a cationic metal), such as sodium nitrite.

Yet, another example of a NO-donor is a corticosteroid, linked to an NO-releasing moiety. An example of such NO-donor is a derivative of hydrocortisone, coded “NCX1022”, having the structure:

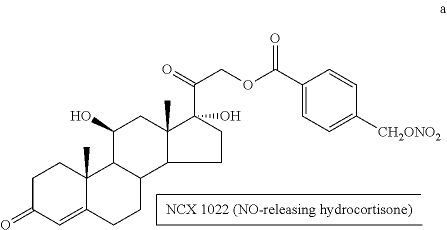

Any other compounds which are capable of releasing NO within the skin layers upon application to the skin are suitable as an NO- precursor or an NO-donor in accordance with the present invention.

Due to the above-mentioned attributes of NO, it can be beneficial, when it is present in the skin in biologically-effective concentrations, it can have beneficial effects on skin disease or condition. For example, NO has antimicrobial properties, i.e., antibacterial, antifungal, and antiviral, properties and thus it has the potential to be used in the treatment of infections of the skin, including but not limited to: bacterial infections, such as cellulitis, impetigo, boils and leprosy; viral infections, such as shingles (herpes zoster), chickenpox, Molluscum contagiosum, warts, measles, hand, foot, and mouth disease; fungal infections, such as athlete's foot, yeast infection, ringworm, nail fungus and oral thrush, and diaper rash.

The symptoms of a skin infection vary depending on the type. Common symptoms include redness of the skin and a rash. People with infection may also experience other symptoms, such as itching, pain, and tenderness. Signs of a severe infection include pus, blisters, skin sloughing, breakdown, dark, necrotic-appearing skin, or skin that becomes discolored and painful.

Acne is a disease that involves an infection by the anaerobic bacterial species Cutibacterium acnes (also named Propionibacterium acnes); and rosacea is also associated with microbial infections (e.g., by Bacillus oleronius).

Additional skin conditions that may involve microbial infection include, in a non-limiting fashion, atopic dermatitis, eczema and psoriasis.

Hence, the topical administration of a composition comprising a sufficient amount of an NO-donor has the potential of treating any of the above skin infections and/or their respective symptoms.

Likewise, the topical administration of an NO-donor or NO-donor has the potential of treating certain symptoms of the skin, which are considered “cosmetic”.

Dry skin is the result of impairment of the water-loss barrier of the skin, which is typical to damaged skin, such as in the event of atopic dermatitis, as well as ageing skin. Topical treatment of patients with atopic dermatitis with an arginine hydrochloride has been shown to significantly increase urea in the stratum corneum as well as an increase in skin moisture, and a consequent improvement of the clinical symptoms of dry skin. One of the most disturbing consequences of dry skin is the formation of wrinkles, which can be abundant across the whole-body surface, with special emphasis on the face, throat and chest, and thus, preventing the impairment of skin barrier function can further impede the formation of wrinkles. Furthermore, as NO induces peripheral blood flow, it can mobilize increased levels of oxygen and nutrients to skin layers, thereby facilitating reconstruction of the tissue and reduction of wrinkle appearance.

As noted above, high concentrations of NO in the skin can cause heating of the skin and inflammation, associated with skin redness (erythema), swelling (oedema) and a burning sensation. Such are considered untoward effects, which may prohibit the use of products by people in need, despite the potential benefits.

### Counter-NO Agent

We surprisingly discovered that combining an NO-donor with an agent that inhibits NO can retain the beneficial effects of NO, while reducing the unwanted side effects. Such an agent is termed herein a “counter-NO agent”.

In an embodiment of the present invention, the counter-NO agent is an agent that inhibits the activity of the enzyme nitric oxide synthase (“NOS”). There are three isoforms of NOS: the brain or neuronal form (“nNOS”), the endothelial form (“eNOS”), and the inducible form (“iNOS”). The form of NOS that is more prevalent in the skin is eNOS, and examples for eNOS inhibitors include, without limitation, 1-N^{G}-nitroarginine (NArg), NG-Nitro- L-Arginine Methyl Ester (L-NAME), and NG-monomethyl-l-arginine (1-NMMA), the methyl ester of NG-nitro-L-arginine (NAME), L-N^{G}-Nitroarginine (NNA), NG-amino-L-arginine (NAA), N^{G}.N^{G}-dimethyl-arginine (asymmetric dimethylarginine, ADMA). S-Ethylisothiouronium diethylphosphate (brand name Difetur) is an additional exemplary eNOS inhibitor, that was shown to be safe for topical application.

SDMA (NG,NG′-Dimethyl-L-arginine), L-Thiocitrulline, S-Methyl-L-Thiocitrulline, Agmatine (1-Amino-4-guanidinobutane), L-NIO Nδ-(Iminoethyl)-L-ornithine, Vinyl-L-NIO, Ethyl-L-NIO, 7-NI (7-Nitroindazole), 7-NI-Br (7-Bromonitroindazole), (1-[2-(Trifluoromethyl)phenyl-imidazole), S-(2-Aminoethyl) isothiourea, Methylene blue, [1H41,2,4]Oxadiazole[4,3-a]quinoxalin-1-one] (from: Pharmaceuticals (Basel). 2010 Jan. 3(1): 273-299. Nitric Oxide Synthase Inhibitors as Antidepressants by Gregers Wegener, and Vallo Volke)

Yet, another way to decrease the activity of eNOS is to suppress the expression of eNOS by cells, thereby limiting its synthesis and reducing the amount on NO in the tissue. A nonlimiting example is nicotinamide, a form of vitamin B3, that reduces NOS protein expression and decreased NO release from the cells.

Of note, nicotinamide is known to exert a plurality of beneficial effects when administered to the skin, including, without limitation, the correction of light-induced skin ageing (it helps protect from UV and blue light damage, reduces the appearance of lines and wrinkles, improves the elasticity of the skin, rebalances uneven skin tone and reduces discoloration; it helps restore skin barrier function and reduces trans-epidermal water loss, which is the underlying factor for induces skin ageing.

Nicotinamide has also been demonstrated to be effective in the prevention and/or treatment of skin diseases, including but not limited to treating acne and rosacea, alleviating pruritus (itch), and it has also been shown to have a chemopreventative role in nonmelanoma skin cancer.

Any salts, isomers, analogs and derivatives of nicotinamide that have a counter-NO property are suitable in the context of the present invention.

### Concentrations of the NO-Donor and the Counter-NO Agent in the Composition

The concentration of the NO-donor in the topical composition of the present invention is determined by the amount that is sufficient to elevate the level of NO in the skin, upon topical application of the composition. Since measuring NO levels in humans is impractical, such elevation can be observed by the appearance of skin redness (erythema), swelling (oedema) a tingling sensation and a burning sensation, which is intensified as the concentration goes up. Furthermore, the occurrence of elevated NO can be noticed by its desirable cosmetic outcomes such as enhanced radiance and reduction of wrinkles.

For example, in the case of nitroglycerine, 2% is insufficient. At higher concentrations, from about 4%, a slight burning sensation is noticed. This sensation intensifies as the nitroglycerine concentration increases. Thus, in certain embodiments, the concentration of nitroglycerine as NO-donor is between about 4% - about 12%; and in other embodiments it can be from about 4%-8%, or from about 8%-12%.

In the case of arginine, 5% is insufficient. At higher concentrations, from about 6%, a slight burning sensation is noticed, and it is associated with a tingling sensation. This sensation intensifies as the arginine concentration increases. Thus, in certain embodiments, the concentration of arginine as NO-donor is at least about 6%. For example, between about 6% and about 25%. In other embodiments, the concentration of arginine can be from about 6% to about 8%, or from about 8% to about 10%, or from about 10% to about 14%, or from about 14% to about 18%, or from about 18% to about 25%, or from about 8% to about 25%, or from about 6% to about 12%, or from about 8% to about 12%, or from about 8% to about 10%.

Likewise, in the case of citrulline, in certain embodiments, the concentration of citrulline is between about 4% and about 20%. In other embodiments the concentration of citrulline can be from about 4% to about 6%, or from about 6% to about 10%, or from about 6% to about 8%, or from about 10% to about 14% or from about 14% to about 20%.

The concentration of the counter-NO agent is determined so that the skin redness (erythema), swelling (oedema) and burning sensation, induced by the NO-donor is decreased or eliminated. For example, in certain cases, where the counter-NO agent is nicotinamide, 0.5% is insufficient. In certain embodiments, the concentration of nicotinamide as a counter-NO agent is at least about 0.25%, at least about 0.5%, at least about 0.6%, at least about 0.7%, at least about 0.8%, at least about 0.9%, or at least about 1%. In certain embodiments, the concentration of nicotinamide as a counter-NO agent is between about 0.25% and up to about 10%. For example, between about 1% and about 2%; and in other embodiments it can be from about 2% to about 4%, or from about 4% to about 6%, or from about 6% to about 8%, or from about 8% to about 10%, or from about 0.25% to about 4%.

In an observatory trial, it was observed that when subjects were topically applied with a composition comprising 10% arginine and 2% nicotinamide, a few subjects reported a mild warming sensation or a slight tingling sensation, while others did not experience any such sensation. Of note, the very slight warmth of the skin or the slight tingling sensation were not considered prohibitive by the subjects, who said that “it feels like the product works”.

Thus, in certain embodiments, the ratio between the NO-donor and the counter-NO agent in the topical composition is between about 1:2 and 8:1; and in other embodiments it can be between about 1:2 and 1:1, or between about 1:1 and 2:1, or between about 2:1 and 4:1, or between about 4:1 and 8:1.

Yet, in an additional embodiment, when the NO-donor is arginine and the counter-NO agent is nicotinamide, based on the above range of concentrations of the arginine of about 6% to about 25% and the corresponding range for nicotinamide is from about 0.25% to 10%, the ratio between the NO-donor and the counter-NO agent in the topical composition is between about 6:10 and about 100:1. In other embodiment, the ratio between the NO-donor and the counter-NO agent in the topical composition is about 100:1 to about 2.5:1. For example, the ratio between the NO-donor and the counter-NO agent is about 40:1 to about 2.5:1, or about 10:1 to about 2.5:1, or about 6:1 to about 2.5:1, or about 5:1 to about 2.5:1, or about 4:1 to about 2.5:1. For example, the composition of the present invention can contain about 10% arginine and 0.25% nicotinamide. Yet, in additional examples, the composition of the present invention can contain about 10% arginine and about 2% nicotinamide, or about 8% arginine and about 2% nicotinamide, or about 10% arginine and about 4% nicotinamide, or about 12% citrulline and about 2% nicotinamide, or about 10% citrulline and about 2% nicotinamide.

In an embodiment, when the NO-donor is arginine or a salt, isomer, analog, or derivative thereof and the counter-NO agent is nicotinamide or a salt, isomer, analog or derivative thereof, the ratio between the NO-donor and the counter-NO agent in the topical composition is between about 2:1 and 8:1; and in other embodiments it can be between about 2:1 and 4:1, or between about 4:1 and 6:1, or between about 6:1 and 8:1.

In an embodiment, the concentration of arginine is between about 7.5% and 12.5% and the concentration of nicotinamide is between about 1% and 10%. In an additional embodiment, the concentration of arginine is about 10% and the concentration of nicotinamide is about 2%. In other embodiments, the concentration of arginine is between about 7.5% and 12.5% and the concentration of nicotinamide is between about 0.25% and about 5%, or between about 0.25% and about 4%.

### Synergistic Effect

In certain cases, the counter-NO agent, beyond its NO-suppression effects, has beneficial effects on the target site of application.

One example is nicotinamide and its salts, isomers, analogs and derivatives, which possess therapeutic effects of its own, as laid out above. In such a case, the therapeutic effect of the NO-donor (such as arginine) and the therapeutic effects of the counter-NO agent (such as nicotinamide) are combined synergistically to achieve improved results, which are better than each agent alone; and without the inherent side effects of the NO-donor in its high concentration.

### Tingling Effect

As noted above, high concentrations of an NO-donor can induce skin redness (erythema), swelling (oedema) and a burning sensation, which can render such composition intolerable. The combination of said NO-donor with a counter-NO agent reduces the intensity of these side effects and in many cases, a slight tingling effect is noticed, which is well tolerated and even considered a positive sign by the subject applying such a composition. In our human tests, several subjects said that “it feels like the product works”, continued to use the product and experienced positive therapeutic results.

Thus, in an embodiment, the invention relates to a topical composition, comprising an NO-donor and a counter-NO agent, which, upon topical application exerts a tolerable tingling effect.

### Additional Therapeutic Agent

Several conditions involve a combination of etiological factors, some of which are affected by NO; and other etiological factors that require an additional therapeutic modality. For example, psoriasis may be treated by NO, as well as a steroid drug, and therefore combined treatment would be beneficial. Likewise, acne, which involves a microbial infection, excessive keratin production, excessive sebum production and inflammation, can benefit from treatment with a combination NO, and an additional therapeutic agent, selected from the group consisting of an anti-inflammatory agent, an antibiotic agent, a sebostatic agent and a keratolytic agent. Hence, in many cases, the inclusion of an additional therapeutic agent in the composition contributes to the clinical activity of NO.

Suitable additional therapeutic agents include but are not limited to active herbal extracts, herbal oils, herbal tinctures, acaricides, age spot and keratose removing agents, allergen, analgesics, antiacne agents, antiallergic agents, antiaging agents, anti-bacterials, antibiotics, antiburn agents, anticancer agents, antidandruff agents, antidepressants, anti-dermatitis agents, anti-edemics, antihistamines, anti-helminths, anti-hyperkeratolyte agents, anti-inflammatory agents, anti-irritants, anti-lipemics, antimicrobials, antimycotics, antiproliferative agents, antioxidants, anti-wrinkle agents, anti-pruritics, anti-psoriatic agents, anti-rosacea agents anti-seborrheic agents, antiseptic, anti-swelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, local anesthetics, metal oxides, mitocides, neuropeptides, non-steroidal anti-inflammatory agents, oxidizing agents, pediculicides, photodynamic therapy agents, retinoids, scabicides, self-tanning agents, skin whitening agents, vasoconstrictors, vasodilators, vitamins, vitamin D derivatives, wound healing agents and wart removers. As is known to one skilled in the art, in some instances a specific active agent may have more than one activity, function or effect.

In one or more embodiments, the herein composition is free or substantially free of at least one of: choline chloride, magnesium chloride and sodium chloride. In one or more embodiments, the herein composition is free or substantially free of at least one of: choline chloride (10% w/v), magnesium chloride (5% w/v), and sodium chloride (5% w/v). In one or more embodiments, the herein composition includes less than 10% w/v of choline chloride, less than 5% w/v magnesium chloride, and/or less than 5% w/v sodium chloride.

In an embodiment, the additional therapeutic agent is a neurotoxin. In an embodiment, the term “neurotoxin” as used herein refers to a substance that is toxic to the nervous system and that can interfere with the normal functioning of neurons or nerve cells, leading to various neurological effects. The neurotoxin may affect muscle contractions, leading to muscle paralysis. In an embodiment, the neurotoxin is botulinum toxin. Botulinum toxin is a naturally occurring neurotoxin produced by the bacterium Clostridium botulinum. It is one of the most potent neurotoxins known to affect the nervous system. Botulinum toxin is widely used for cosmetic purposes to temporarily reduce the appearance of facial wrinkles and fine lines.

In an embodiment of the invention, the neurotoxin is a neurotoxin-mimetic agent. Neurotoxin-mimetic agents are synthetic or naturally-derived compounds that mimic the effects of neurotoxins. They may have similar effects as neurotoxins but are typically less toxic or harmful. As used herein the neurotoxin-mimetic agents can reduce wrinkles and fine lines. Neurotoxin-mimetic agents are sometimes peptides, which interact with biological targets that are involved in the process of muscle contraction.

An example of such an agent is acetyl hexapeptide-3 (AC-gly glu-met-gln-arg-arg-NH2), which inhibits SNARE complex formation and catecholamine release. This peptide is currently marketed as an aqueous solution under the commercial name Argireline® and the recommended concentrations of the solution in a topical composition is between about 0.8% and 10%.

Yet, another example is Tripeptide-3 (beta-Ala-Pro-Dab-NH-benzyl×2 AcOH), currently marketed as Syn®-Ake, and the recommended concentrations of the solution in a topical composition is between about 1% and 4%. It is a neurotoxin found in the venom of the temple viper, which causes reversible antagonism of muscular nicotinic acetylcholine receptors (mnAChR) at the postsynaptic membrane. It is proposed to act similarly to Waglerin 1(a polypeptide that is found in the venom of the temple viper).

An additional neurotoxin-mimetic agent is acetyl hexapeptide-1, which is a synthetic peptide composed of the amino acids alanine, arginine, histidine, leucine, phenylalanine and tryptophan, marketed under the name Munapsys™and the recommended concentrations of the solution in a topical composition is between about 2% and 5%. It binds to syntaxin allowing the assembly of the SNARE complex and competes with Munc-18 for its position in syntaxin, obstructing SNARE complex assembly and the release of the acetylcholine (ACh) neurotransmitter into the synapse.

Another exemplary neurotoxin-mimetic is peptide Pentapeptide-3 (Lys-Thr-Thr-Lys-Ser, KTTKS), or its derivative palmitoylated derivative (pal-KTTKS) a competitive antagonist at the acetylcholine postsynaptic membrane receptor currently marketed as Vialox.

As outlined above the various neurotoxin-mimetic agents act through different biological mechanisms of action or bind to different receptors or other biochemical targets.

In an embodiment of the present invention, the additional therapeutic agent is included in the herein composition comprising the NO-donor and the counter-NO agent. Yet in additional embodiments, the herein composition comprising the NO-donor and the counter-NO agent is used in concurrence with another composition comprising one or more of the additional therapeutic agents.

In an embodiment, there is provided a kit of topical compositions, wherein one composition (a first topical composition) comprises the herein composition comprising an NO-donor and a counter-NO agent, and the other composition (a second topical composition) comprises one or more of the additional therapeutic agents.

The second composition may comprise one or more neurotoxins. For example, the second composition may comprise one or more neurotoxin-mimetic agents. For example, the second composition may comprise one neurotoxin-mimetic agent, a combination of two neurotoxin-mimetic agents, or a combination of three neurotoxin-mimetic agents. An exemplary second composition may comprise Argireline® together with Syn®-Ake or Pemtapeptide-3 or Munapsys™ or Vialox. An exemplary second composition may comprise a combination of three neurotoxin-mimetic agents, such as, Argireline® together with two agents, selected from Syn-Ake®, Pemtapeptide-3, and Munapsys™ and Vialox. In an embodiment, salts, isomers, analogs and derivatives of the neurotoxin-mimetic agents are further herein contemplated.

In an embodiment, the kit of topical compositions includes: a first topical composition comprising arginine and nicotinamide, in the concentrations specified above, and a second composition comprising one or more of the additional therapeutic agents. The two compositions of the kit can be applied to the skin concurrently, or serially, for example, the first composition in the morning and the second composition in the evening.

The use of such a kit is advantageous, since the first composition has, among its other properties, the effect of enhancing blood circulation in the skin, which may facilitate the efficient skin penetration of the additional therapeutic agent. This advantage is particularly relevant when the additional therapeutic agent is one or more neurotoxin-mimetic peptides, which require penetration enhancement in order to exert their optimal effect.

### Forms of the Composition

The composition of the resent invention comprises a vehicle, an NO-donor and a counter-NO agent. While the composition may have various rheological characteristics, the following non-limiting examples of forms of the composition are provided for demonstration purposes.

In an embodiment, the composition is liquid. A liquid composition is flowable.

In an embodiment, the composition is an aqueous liquid, wherein the NO-donor and the Counter-NO agent are either in solution or in suspension.

In additional embodiments, the composition is semi-solid. In certain embodiments the composition has a viscosity of more than about 5,000 Cps and it can have a viscosity selected from the group of: between about 5,000 Cps and about 100,000 Cps; between about 5,000 Cps and about 20,000 Cps;

between about 20,000 Cps and about 60,000 Cps; and between about 60,000 Cps and about 100,000 Cps.

In an embodiment, the composition is a gel. The viscosity of the gel can be attained using customary polymeric or gelling agents. Exemplary polymeric or gelling agents include, in a non-limiting manner, naturally-occurring polymeric materials, such as locust bean gum, sodium alginate, sodium caseinate, egg albumin, gelatin agar, carrageenan gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinyl pyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses. Polyethylene glycol, having molecular weight of 1000 or more (e.g., PEG 1,000, PEG 4,000, PEG 6,000 and PEG 10,000) also have gelling capacity and while they are considered herein as “secondary polar solvents”, as detailed herein, they are also considered polymeric agents.

Mixtures of the above polymeric agents are contemplated.

In an embodiment, the composition is an aqueous gel, i.e., a gel that contains water, wherein the NO-donor and the Counter-NO agent are either in solution or in suspension.

Yet, in further embodiments, the composition is an emulsion, or micro-emulsion, or a nano-emulsion, which includes an aqueous phase and an organic carrier phase. Examples of topical dosage forms that comprise an emulsion are creams, lotions and emulsion-based sprays and foams.

Yet, in an additional embodiment, the composition is intended to be applied in the form of a stick such as lipstick. In the past, lipsticks have mainly focused on bringing decorative benefits (color shade, gloss) to the lips. Our lipsticks provide functional benefits such as rejuvenation, increasing the natural vitality and provide nutrients and therapeutic agents.

In an embodiment, the agents may comprise inhibitors of the enzyme hyaluronidase, and thus will slow down or eliminate the enzymatic degradation of the injected Hyaluronic acid and other lips volumizing or plumping agents.

In contrast to creams (see forms and compositions), lipsticks comprise pigments (colorants), oils and waxes. Many oils can be used in lipsticks, such as castor oil, mineral oils, and hydrogenated vegetable oils. The oils viscosity ranges from liquid to near wax-like, and they play a role of dispersant for colorants as well as cohesion enhancer in lipsticks. Typical oil concentrations range from 6 to 10%.

The most used waxes in lipsticks are beeswax and Carnauba wax. Waxes are used to increase the viscosity of a lipstick and balance the effects of oils and esters. Waxes are harder ingredients, and they raise the melting point of a formulation. This control in the melting temperature of the lipstick also controls the payoff of a lipstick, which is the amount of product transferred from the lipstick to the lips. Typical wax concentrations range from 8 to 18%.

Finally, polymers may be added to impart film-forming properties to lipsticks as well as to assist the formed film cohesion. Another benefit of polymers is the enhancement of wear-resistance. Usually, high-MW polymers are used for film adhesion and flexibility to follow the movements of the lips while lower-MW branched polymers can create a three-dimensional local network inside the film and traps colorant. Examples of polymers: acrylate/C12-22 alkylmethacrylate copolymers

The organic carrier is selected from a hydrophobic organic carrier (also termed herein “hydrophobic carrier”), an emollient and mixtures thereof.

In one or more embodiments, the hydrophobic carrier is an oil, such as mineral oil. According to one or more embodiments, hydrophobic carriers are oils originating from plant, marine or animal sources. By way of example, the plant oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

Suitable hydrophobic carriers also include polyunsaturated oils that contain, for example omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which may contribute to the therapeutic benefit of NO.

In the context of the present invention, oils that possess therapeutically-beneficial properties are termed “therapeutically active oil”.

Silicone oils may also be used. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Silicone oils are also considered therapeutically active oil, due to their barrier retaining and protective properties.

The organic carrier may contain a mixture of two or more of the above hydrophobic carriers in any proportion.

A further class of organic carrier includes “emollients” that have a softening or soothing effect, especially when applied to body areas, such as the skin and mucosal surfaces. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof Surface-active agents (also termed “surfactants”) include any agent linking oil and water in the composition, in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average).

According to one or more embodiments of the present invention, the surface-active agent has a hydrophilic lipophilic balance (HLB) between about 9 and about 14, which is the required HLB (the HLB required to stabilize an 0/W emulsion of a given oil) of hydrophobic carriers or oils. Thus, in one or more embodiments, the composition contains a single surface-active agent having an HLB value between about 9 and 14, and in one or more embodiments, the composition contains more than one surface active agent and the weighted average of their HLB values is between about 9 and about 14. Yet, in other embodiments, when a water in oil emulsion is desirable, the composition contains one or more surface active agents, having an HLB value between about 2 and about 9.

The surface-active agent is selected from anionic, cationic, nonionic, zwitterionic, amphoteric and ampholytic surfactants, as well as mixtures of these surfactants. Such surfactants are well known to those skilled in the therapeutic and cosmetic formulation art. Nonlimiting examples of possible surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines.

In an embodiment, the composition of the present invention is a serum.

In an embodiment, the composition of the present invention is a foam.

In an embodiment, the composition of the present invention is attached to a mask, to be applied to the skin for an occlusive treatment; and to be removed following an extended period of application.

### Treatment/Therapy

The terms “therapy” and “treatment” as used herein interchangeably, cover any treatment of a disease or disorder or a cosmetic condition, and includes, for example:
curing the disease or disorder or cosmetic condition;
preventing the disease or disorder or cosmetic condition from occurring in a subject which may be predisposed to the disease but has not yet been diagnosed as having it;
inhibiting the disease or disorder or cosmetic condition;
relieving the disease or disorder or cosmetic condition;
causing regression of the disease or cosmetic condition;
Improving the effect of a therapeutic agent, which is known to affect the disease or disorder or cosmetic condition;
providing a beneficial immunological effect;
improving the quality of life of a subject afflicted by a disease or disorder; and, in the case of cosmetic treatment.
cleansing, beautifying, promoting attractiveness, or altering the appearance without affecting the body's structure or functions in the following, some non-limiting examples and experiments are described in detail. This invention is not limited to these examples and experiments. Many variations will suggest themselves are within the full intended scope of the appended claims.

### Fields of Applications

By including an appropriate NO-donor, which evolves NO, the composition of the present invention is useful in treating a patient having any one of a variety of dermatological disorders (also termed “dermatoses”), such as classified, in a non-limiting exemplary manner, according to the following groups: dermatitis, including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash; bacterial infections, including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma; fungal infections, including dermatophyte infections, yeast Infections; parasitic infections, including scabies, pediculosis, creeping eruption; viral infections; disorders of hair follicles and sebaceous glands, including acne, rosacea, perioral dermatitis, hypertrichosis (hirsutism), alopecia, male pattern baldness, alopecia areata, alopecia universalis and alopecia totalis; pseudofolliculitis barbae, keratinous cyst; scaling papular diseases, including psoriasis, pityriasis rosea, lichen planus, pityriasis rubra pilaris; benign tumors, including moles, dysplastic nevi, skin tags, lipomas, angiomas, pyogenic granuloma, seborrheic keratoses, dermatofibroma, keratoacanthoma, keloid; malignant tumors including basal cell carcinoma, squamous cell carcinoma, melanoma, paget's disease of the nipples, kaposi's sarcoma; reactions to sunlight, including sunburn, chronic effects of sunlight, photosensitivity; bullous diseases, including pemphigus, bullous pemphigoid, dermatitis herpetiformis, linear immunoglobulin A disease; pigmentation disorders, including hypopigmentation, vitiligo, albinism, post-inflammatory hypopigmentation, post-inflammatory hyperpigmentation, melasma, chloasma, drug-induced hyperpigmentation; disorders of cornification, including ichthyosis, keratosis pilaris, calluses, corns, actinic keratosis; pressure sores; disorders of sweating; inflammatory reactions including drug eruptions, toxic epidermal necrolysis; erythema multiforme, erythema nodosum, granuloma annulare.

### The NO-Donors

In an embodiment, the NO-donor is arginine.

In an embodiment, when the NO-donor is arginine and the counter-NO agent is nicotinamide, the composition of the present invention is useful in treating a skin condition, selected from the group consisting of a skin infection, acne, rosacea, atopic dermatitis, eczema and psoriasis.

In an embodiment, the NO-donor is citrulline.

In an embodiment, when the NO-donor is citrulline and the counter-NO agent is nicotinamide, the composition of the present invention is useful in treating a skin condition, selected from the group consisting of a skin infection, acne, rosacea, atopic dermatitis, eczema and psoriasis.

In additional embodiments, when the NO-donor is arginine and the counter-NO agent is nicotinamide, the composition of the present invention is useful in treating the signs of ageing skin, including light-induced skin ageing, increased transepidermal water loss (TEWL), skin dryness, fine lines, wrinkles, hyperpigmentation and skin discoloration.

Yet, in additional embodiments, when the NO-donor is arginine and the counter-NO agent is nicotinamide, the composition of the present invention is useful for application in concurrence with the administration of a neurotoxin, such as botulism toxin A (“BTX”). It has been reported that intradermal injection of botulinum toxin A eliminates cutaneous vasodilation (Brett et al., TEMPERATURE 2017; 4/1:41-59); and by doing so, BTX reduces the supplies of oxygen and nutrients to the skin tissues. It has additionally been found that the topical administration of NO-donors potentiates the muscle relaxation effect of BTX (Lysy at al., Gut 2001;48:221-224). Therefore, the composition of the present invention, which releases NO into the skin, can facilitate increased supplies of oxygen and nutrients to the skin tissues, augment the effect of BTX and prolong the duration of the effect. The composition can be used prior to BTX injection and then on a continuous basis following BTX injection. This composition can also be used to alleviate the inflammation that is induced following BTX injections.

The composition of the present invention, containing an NO donor, can further be useful in alleviating the atrophy of the skin due to BTX Injection.

The compositions of the present invention are useful in the therapy of non-dermatological disorders, which respond to topical/transdermal delivery of an active agent. By way of example, such disorders include localized pain and/or in general, as well as joint pain, muscle pain, back pain, rheumatic pain, arthritis, osteoarthritis and acute soft tissue injuries and sports injuries. Other disorders of this class include conditions which respond to hormone therapy, such as hormone replacement therapy, transdermal nicotine administration, and other respective disorders, known in the art of drug delivery.

Thus, the compositions of the present invention are useful in treating a patient having any one of a variety of gynecological disorders, such as classified, in a non-limiting exemplary manner, according to the following groups: pelvic pain, including premenstrual syndrome (PMS), mittelschmerz (severe midcycle pain due to ovulation), dysmenorrhea (pain related to the menstrual cycle), endometriosis, ectopic pregnancy, ovarian cysts and masses, acute pelvic inflammatory disease, pelvic congestion syndrome and vulvodynia; vulvovaginal infections, including bacterial vaginosis, candidal vaginitis, trichomonas vaginalis, herpes simplex genital ulcers and warts, pelvic inflammatory disease, cervicitis, acute and chronic salpingitis; endometriosis; gynecological neoplasms, including endometrial Cancer, ovarian cancer, cervical cancer, vulvar cancer, vaginal cancer, fallopian tube cancer and gestational trophoblastic disease; benign tumors; sexually transmitted diseases; sexual dysfunction disorders that respond to pharmacological therapy, including sexual arousal disorder, female orgasmic disorder, dyspareunia and vaginismus; and various gynecological disorders that respond to hormonal therapy.

Rectal applications include, for example, anal abscess/fistula, anal cancer, anal warts, hemorrhoids, anal and perianal pruritus, soreness, excoriation, perianal thrush, anal fissures, fecal incontinence, constipation, Crohn's disease, inflammatory Bowel's disease and polyps of the colon and rectum.

The compositions of the present invention are further useful for intra-vaginal and rectal treatment of sexually-transmitted and non-sexually-transmitted infectious diseases (STDs).

In one or more embodiments, the invention provides a method of treatment of a disease or disorder of the skin, mucosal membranes, the anum, the rectum, the GI system, the vagina, the penile urethra, the eye, the respiratory system, including the oral cavity, the nasal cavity, the sinuses, the pharnix, the larynx, the trachea, the bronchus and the lungs, the dental system and the ear canal, comprising topical administration of the composition of the present invention, whereby one or more NO-donors, in a therapeutically effective concentration is administered topically to the afflicted area.

As used in this written description, the singular forms “a,” “an” and “the” include express support for plural referents unless the context clearly dictates otherwise. Thus, for example, reference to “a gas” includes a plurality of such gases.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in this written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein.

Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein. The term “coupled,” as used herein, is defined as directly or indirectly connected in an electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, comparative terms such as “increased,” “decreased,” “better,” “worse,” “higher,” “lower,” “enhanced,” “maximized,” “minimized,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art. For example, a process that has an “increased” therapeutic effect or result can refer to improved results or efficacy attained by the process as compared to a similar or different process intended for treatment of the same condition or experience.

As used herein, the term “isomer” refers to each of two or more compounds that have the same molecular formula but differ in the arrangement of atoms within the molecule. The herein term encompasses isomers that maintain or even enhance the functional effect claimed by the herein compounds.

As used herein the term “analog” refers to a compound or molecule that is structurally related to another compound but differs in one or more components or functional groups. The herein term encompasses analogs that maintain or even enhance the functional effect claimed by the herein compounds.

As used herein the term “derivative” refers to a compound that is derived from another compound by making specific modifications or substitutions. Derivatives are created by altering the structure or functional groups of the parent compound while retaining some of its key characteristics. The herein term encompasses derivatives that maintain or even enhance the functional effect claimed by the herein compounds.

As used herein the term “cosmetically or pharmaceutically acceptable salts” refer to derivatives of the herein compounds, wherein the parent compound is modified by making non-toxic acid or base addition salts thereof, and further refers to cosmetically or pharmaceutically acceptable solvates, including hydrates, of such compounds and such salts.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. In some embodiments, the term “substantially” means practically meaningful or statistically significant. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. As used herein the term “about” refers to ±20%, ±10%, or ±5%. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The term “subject” refers to a human or a non-human mammal.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLES

### Example 1. Cream compositions

### Composition #1 comprising L-arginine and nicotinamide (niacinamide)

| | | | | % BY |
|---|---|---|---|---|
| PHASE | INGREDIENT | FUNCTION | INCI DESIGNATION | WEIGHT |
| A | WATER | Diluent | WATER (AQUA) | 22.1 |
| A | NA₂EDTA | Chelating agent | DISODIUM EDTA | 0.1 |
| A | VEEGUM T | Thickener | MAGNESIUM | 1.0 |
| | | | ALUMINUM SILICATE | |
| A | KELTROL CG- | Thickener | XANTHAN GUM | 0.35 |
| | SFT | | | |
| A | GLYCERIN | Humectant | GLYCERIN | 0.5 |
| | 99.7% | | | |
| A | MPDIOL | Humectant | METHYLPROPANEDIOL | 3.0 |
| | GLYCOL | | | |
| B | STEARIC ACID | Emulsion stabilizer | STEARIC ACID | 6.0 |
| B | CERASYNT IP | Emulsion stabilizer | GLYCOL STEARATE | 2.0 |
| | | | STEARAMIDE AMP | |
| B | XIAMETER | Emollient | CYCLOPENTASILOXANE | 3.5 |
| | PMX-0245 | | | |
| B | °RANSIL GCM- | Skin feel | CYCLOPENTASILOXANE | 4.5 |
| | 5 | | POLYSILICONE•11 | |
| B | LIPOWAX 0 | Emulsifier | CETEARYL ALCOHOL | 1.0 |
| | | | CETEARETH-20 | |
| B | LIPEX SHEA | Emollient | SHEA) BUTTER | 1.0 |
| B | JEECHEM OP | Emollient | ETHYLHEXYL | 2.5 |
| | | | PALMITATE | |
| B | (AVOCADO | Emollient | PERSA GRATISSIMA | 0.5 |
| | OIL) | | (AVOCADO) OIL | |
| B | TENOX BHT | Antioxidant | BHT | 0.05 |
| B | VITAMIN E | Antioxidant | TOCOPHERYL ACETATE | 0.05 |
| | ACETATE | | | |
| C | SODIUM | pH adjustor | SODIUM HYDROXIDE | 0.76 |
| | HYDROXIDE | | WATER | |
| | 20% | | | |
| D | WATER | Diluent | WATER (AQUA) | 5.0 |
| D | (GLYCINE) | Skin condition | GLYCINE | 0.1 |
| | | agent | | |
| D | L-PROLINE | Improves skin | PROLINE | 0.1 |
| | | elasticity & | | |
| | | collagen | | |
| | | production | | |
| D | L-LEUCINE | Skin conditioning | LEUCINE | 0.1 |
| 0 | L-ISOLEUCINE | Skin conditioning | ISOLEUCINE | 0.1 |
| D | NIACINAMIDE | Counter-NO agent | NIACINAMIDE | 2.00 |
| I) | L-VALINE | Moisturizing and | VALINE | 0.10 |
| | | relaxing agent | | |
| E | WATER | Diluent | WATER (AQUA) | 5.00 |
| E | L-ARGININE | NO-donor | ARGININE | 10.00 |
| E | CITRIC ACID | pH adjustor | WATER + CITRIC ACID | 10.5 |
| | 50% | | | |
| F | Primalhyal 300 | Hydrating and | Sodium Hyaluronate | 2.0 |
| | | repairing | | |
| F | URSOLIC ACID | Skin firming | SODIUM URSOLATE | 0.20 |
| | SODIUM SALT | | (AND) SODIUM | |
| | | | OLEANOLATE | |
| F | ACTIPHYTE OF | Antioxidant | BUTYLENE GLYCOL, | 0.05 |
| | GREEN TEA | | CAMELLIA SINENSIS | |
| | BG5OP | | LEAF EXTRACT | |
| F | MAGNESIUM | Antioxidant | MAGNESIUM | 0.05 |
| | ASCORBYL | | ASCORBYL PHOSPHATE | |
| | PHOSPHATE | | | |
| F | ACTIPHYTE OF | Anti-inflammatory | BUTYLENE GLYCOL | 0.10 |
| | IRISH MOSS | | WATER CHONDRUS | |
| | BG5OP | | CRISPUS f | |
| | | | CARRAGEENAN) | |
| | | | EXTRACT | |
| F | ACTIPHYTE OF | Anti-inflammatory | BUTYLENE GLYCOL | 0.10 |
| | NUTMEG | | WATER MYRISTICA | |
| | BG5OP | | FRAGANS (NUTMEG) | |
| | | | EXTRACT | |
| F | FRESH & | Fragrance | FRAGRANCE | 0.20 |
| | CLEAN #28353 | | | |
| F | MIKROKILL | Preservative | PHENOXYETHANOL | 1.25 |
| | COS | | CAPRYLYL GLYCOL | |
| | | | CHLORPHENESIN | |
| F | FLORAL | Aromatic water | CHAMOMILLA | 15.00 |
| | ESSENTIAL | | RECUTITA | |
| | WATER H5464 | | (MATRICARIA) FLOWER | |
| | | | WATER | |
| G | SODIUM | pH adjustor | SODIUM HYDROXIDE | 1.4 |
| | HYDROXIDE | | WATER | |
| | 20% | | | |
| Total | | | | 100 |

### Preparation Procedure

PHASE A
   Combine water and EDTA. Mix to dissolve EDTA. Slowly disperse veegum plus into agitating water.
   Slowly disperse KELTROL into phase a. Begin heating batch to 75° C. Add remaining phase A ingredients.
   Homogenize phase A for 2 minutes at approximately 3500 rpm
PHASE B
   Combine phase B ingredients and heat with mixing to 75° C.
   Slowly add phase B to phase A with mixing phase C use phase C to adjust pH of batch to 6.3 - 7.5 cool batch to 40° C.
PHASE D
   Combine phased ingredients glycine in water first. Mix until powders dissolve
   Add remaining ingredients mixing one by one until uniform, add the L-leucine last, then add to batch with mixing
PHASE E
   Combine ingredients and water. Mix until uniform.
   Use citric acid to adjust pH to 6.74
   Adjusting the pH will bring this phase to a clear solution
   Add to batch with mixing
PHASE F
   Add phase F ingredients to batch one at a time with mixing for 2 minutes in between each addition
PHASE G
   Adjust the pH of the batch to 6.9-7.0 by adding sodium hydroxide solution
   Cool batch to 30° C.
PHASE H
   Slowly add phase H to the batch.
PACKAGE

pH: 6.15

VISCOSITY (CPS): about 60,000

### Composition #2 Comprising L-arginine (without Niacinamide)

| | INGREDIENT (TRADE | | | % BY |
|---|---|---|---|---|
| PHASE | NAME) | FUNCTION | INCI DESIGNATION | WEIGHT |
| A | WATER | Diluent | Water (aqua) | 22.13 |
| A | NA2EDTA | Chelating agent | Disodium EDTA | 0.10 |
| A | VEEGUM T | Thickener | Magnesium aluminum | 1.00 |
| | | | silicate | |
| A | KELTROL CG-SFT | Thickener | Xanthan gum | 0.35 |
| A | GLYCERIN 99.7% US P - | Humectant | Glycerin | 0.50 |
| | DEWOLF | | | |
| A | MPDIOL GLYCOL | Humectant | Methylpropanediol | 3.00 |
| B | TRIPLE PRESSED | Emulsion stabilizer | Stearic acid | 8.00 |
| | STEARIC ACID • FGK | | | |
| B | CERASYNT IP | Emulsion stabilizer | Glycol stearate stearamide | 2.00 |
| | | | AMP | |
| B | XIAMETER PMX-0245 | Emollient | Cyclopentasiloxane | 3.50 |
| B | °RANSIL GCM-5 | Skin feel | Cyclopentasiloxane | 4.50 |
| | | | polysilicone•11 | |
| B | LIPOWAX 0 | Emulsifier | Cetearyl alcohol | 1.00 |
| | | | ceteareth-20 | |
| B | JEECHEM OP | Emollient | Ethylhexyl palmitate | 2.50 |
| B | L POVOL A (AVOCADO | Emollient | Persa gratissima | 0.50 |
| | OIL) | | (avocado) oil | |
| B | TENOX BHT | Antioxidant | BHT | 0.05 |
| B | VITAMIN E ACETATE - | Antioxidant | Tocopheryl acetate | 0.05 |
| | JEEN | | | |
| C | SODIUM HYDROXIDE | pH adjustor | Sodium hydroxide water | 0.76 |
| | 20% | | | |
| D | WATER | Diluent | Water (aqua) | 5.00 |
| D | ORISTAR GLC | Skin condition agent | Glycine | 0.10 |
| | (GLYCINE) | | | |
| D | L-PROLINE | Improves skin | Proline | 0.10 |
| | | elasticity & collagen | | |
| | | production | | |
| D | L-LEUCINE | Skin conditioning | Leucine | 0.10 |
| 0 | L-ISOLEUCINE | Skin conditioning | Isoleucine | 0.10 |
| I) | L-VALINE | Moisturizing agent | Valine | 0.10 |
| | | and relaxing agent | | |
| E | WATER | Diluent | Water (aqua) | 5.00 |
| E | L-Arginine | NO-donor | Arginine | 10.00 |
| E | CITRIC ACID 50% SOLN | pH adjustor | Water citric acid | 10.94 |
| F | URSOLIC ACID SODIUM | Skin firming | Sodium ursolate (and) | 0.20 |
| | SALT | | sodium oleanolate | |
| F | ACTIPHYTE OF GREEN | Antioxidant | Butylene glycol water | 0.05 |
| | TEA BG5OP | | camellia sinensis leaf | |
| | | | extract | |
| F | VC-PMG - MAGNESIUM | Antioxidant | Magnesium ascorbyl | 0.05 |
| | ASCORBYL PHOSPHATE | | phosphate | |
| F | ACTIPHYTE OF IRISH | Anti-inflammatory | Butylene glycol, chondrus | 0.10 |
| | MOSS BG5OP | | crispus carrageenan | |
| | | | extract | |
| F | ACTIPHYTE OF | Anti-inflammatory | Butylene glycol, myristica | 0.10 |
| | NUTMEG BG5OP | | fragans (nutmeg) extract | |
| F | FRESH & CLEAN #28353 | Fragrance | Fragrance | 0.20 |
| F | MIKROKILL COS | Preservative | Phenoxyethanol caprylyl | 1.25 |
| | | | glycol chlorphenesin | |
| F | ESSENTIAL WATER | Aromatic water | Chamomilla recutita | 15.0 |
| | BLEND N21561 (MOD OF | | (matricaria) flower water | |
| | H5464) | | | |
| G | Sodium hydroxide 20% | pH adjustor | Sodium hydroxide water | 1.41 |
| | TOTAL | | | 100% |

Preparation - similar to Example #1

### Example 2. Treatment of Ageing Skin (Pilot Study #1) Objectives: To determine the impact of Compositions #1 and #3 on middle-aged women after 1 month.

Methods: Two women over the age of 40 volunteered to receive a month supply of Composition #1 and Composition #2. They were asked to use both compositions daily for 28 days on two sides of the face and to provide input through post use questionnaire.

Results: The results are summarized in the following table.

| | Composition #1 | | | |
|---|---|---|---|---|
| | (10% Arginine + | | Composition #2 | |
| | 2% Nicotinamide) | | (10% Arginine) | |
| | Subject | Subject | Subject | Subject |
| Question | 1 | 2 | 1 | 2 |
| Rate the efficacy of each | | | | |
| composition on a scale | | | | |
| of 1-5 (1 = low, 5 = high) | | | | |
| Firming | 4 | 5 | 4 | 4 |
| Lifting | 4 | 5 | 4 | 4 |
| Reduces wrinkles | 4.5 | 4 | 4 | 3.5 |
| Glows the skin | 3 | 3 | 3 | 3 |
| Keeps it even toned? | 3 | 3.5 | 3 | 3 |
| Keeps your skin | 4 | 4 | 3 | 4 |
| moisturized | | | | |
| Relaxes the skin | 4 | 3 | 3 | 3.5 |
| Rate burning sensation | | | | |
| and tingling on a scale | | | | |
| of 1-5 (1 = low, 5 = high) | | | | |
| Burning sensation | 0.5 | 0 | 2 | 3 |
| Tingling | 0.5 | 0.5 | 1 | 1.5 |
| Overall acceptability | High | Good | Moderate | Poor |

### Conclusion:

1. Composition #1, containing 10% arginine +2% nicotinamide was highly effective in firming and lifting the skin and reducing wrinkles. Composition #2, which contained 10% arginine but not nicotinamide was slightly less effective.
2. Composition #2 induced a notable burning sensation, which made it moderately or poorly tolerable by both subjects. By contrast, Composition #1 was highly tolerated, with a slight tingling effect that was not prohibitive for both subjects.

### Example 3. Treatment of ageing skin (study #2)

Objectives: To determine the impact of Composition #1 on middle-aged women after 28 days of continuous use.

Methods: 20 women over the age of 40 in 3 countries (USA, Italy and Israel) volunteered to receive one-month supply of Composition #1 for use as their day cream. They were asked to use the cream daily for 28 days and to provide unguided input (free form)

Results: 13 of the 20 women provided feedback that was summarized in the table below.

| No. | Initials | Highlights | Comment |
|---|---|---|---|
| 1 | K. P. S. | Brings on a skin | With the cream, you can instantly feel your face relax -- a |
| | | tighter, silkier, and | gentle warmth brings on a skin tighter, more silky, and more |
| | | more youthful | youthful -- it is both instant and, persistent. Over the long- |
| | | | term, little by little, a more relaxed appearance seems to |
| | | | settle in where once fine lines had previously entrenched! |
| 2 | C. B. | It far surpasses any | The cream does exactly what its name suggests, it flows onto |
| | | other product that I | your skin effortlessly and immediately you feel a velvety |
| | | have tried! | difference in your skin's texture. I have used the cream for |
| | | | about three months and have noticed a significant reduction |
| | | | in fine lines. My skin has a renewed glow and feels |
| | | | energized and brighter. I can't say enough about the cream's |
| | | | difference”, it far surpasses any other product that I have |
| | | | tried!” |
| 3 | J. C. | I have received a lot | I have been using these face cream for about a year now and |
| | | of compliments on | I could not be happier with the results! I love the sun and |
| | | my skin | never took great care of my skin however the cream seems |
| | | | to have corrected even previous damage. My face actually |
| | | | looks much prettier and over the past few months I have |
| | | | received a lot of compliments on my skin even that I am |
| | | | ‘glowing ‘Most importantly my skin feels better and is |
| | | | definitely softer I highly recommend it as it is a fabulous |
| | | | product and I love my results!” |
| 4 | F. S. | I have never had a | I have used the cream over a period of several months and |
| | | cream actually do | really love it. The first thing I noticed was the really nice |
| | | that before.” | scent---very subtle but lovely. I applied it to my face and felt |
| | | | some tingling immediately. After about 10-15 minutes, I |
| | | | could feel my skin tightening. I have never had a cream |
| | | | actually do that before. It felt almost like a masque. After |
| | | | using it for a couple of days, I no longer felt the tingling, and |
| | | | my skin looked and felt really soft and my face looked rested |
| | | | and refreshed. I am a believer. The cream is a necessity now - |
| | | | I don't want to lose my beautiful skin!” |
| 5 | E. L. | It gives my skin | Yes, I am using the cream . . . |
| | | instant comfort and | I can tell you that it gives my skin instant comfort and |
| | | excellent moisture: | excellent moisture: A cumulative! |
| | | | The absorption is fast in penetrating into my skin, that's |
| | | | IMPORTANT TO ME. |
| | | | NO IRRITATION. |
| | | | I see and feel my skin soft, silky and glowing. |
| | | | SLOWLY I have been leaving aside other creams used |
| | | | before . . . |
| | | | To my eye I see the benefits now and happy with that and I |
| | | | expect more later . . . |
| | | | Again, it's accumulative. |
| 6 | J. C. | The day cream seems | I have been using the face cream and I could not be happier |
| | | to have corrected | with the results! |
| | | even previous | I love the sun and never took great care of my skin however |
| | | damage. My face | the cream seems to have corrected even previous damage. |
| | | actually looks much | My face actually looks much prettier and over the past few |
| | | prettier and over the | months I have received a lot of compliments on my skin |
| | | past few months I | even that I am ‘glowing’ Most importantly my skin feels |
| | | have received a lot of | better and is definitely softer I highly recommend the cream - |
| | | compliments | it is a fabulous product and I love my results!” |
| 7 | M. F. L | It generates a | The day cream is fantastic! I have been using it daily for |
| | | pearlescent, healthy- | about 1 year and it generates a pearlescent, healthy- |
| | | appearing glow to | appearing glow to my skin. I previously used creams from |
| | | my skin. | some internationally known brands that do not compare to |
| | | | this newer, innovative cream. I have recently given this |
| | | | cream to my Mom and sister and their skin looks great. |
| 8 | F. A. | The skin on my face | I have been using the cream for almost three months and I |
| | | was a little | have noticed that by day it creates a very slight overheating |
| | | “wrinkled” . . . after | on my skin, like a pleasant stimulation (slight warmth) of the |
| | | three months of the | epidermis. |
| | | cream it was as if it | It is as if during the day something worked on my skin to |
| | | had been “stretched” | make it more compact and luminous! A feeling never heard |
| | | | before. |
| | | | The skin on my face was a little “wrinkled” . . . after three |
| | | | months of continued use of the cream day and night it was |
| | | | as if it had been “stretched”. I am very happy with the result. |
| | | | My skin is now smooth and bright! |
| 9 | C. R. | With a small amount | I've been using the cream for a few weeks. |
| | | my skin is bright, | I have a very delicate skin and other creams, even if of big |
| | | nourished | brands, have often caused me redness, with this cream it did |
| | | | not happen. This cream evoked very slight tingling, which |
| | | | is very acceptable and actually made me feel like it does |
| | | | something to my skin. |
| | | | The cream does not grease my face, with a small amount my |
| | | | skin is bright, nourished and over time I noticed a more |
| | | | uniform color. Excellent cream as a base for makeup |
| | | | because it does not alter the consistency of foundation. |
| 10 | Y. E. | The feeling is that the | After using the cream, I can definitely say that in regular use |
| | | skin is firm solid, | of the cream the feeling is that the skin is firm solid, smooth, |
| | | smooth, moisturized | moisturized and flexible around the eyes and forehead and it |
| | | and flexible around | is visible! No other cream to this day has given me that |
| | | the eyes and forehead | feeling. |
| | | | I liked the slight tingling sensation. It feels like the product |
| | | | works. |
| 11 | E. D. | | For about a month now I use the cream. I have experienced |
| | | | many types of creams in my quite few years, including |
| | | | expensive creams of prestigious brands, and I can say |
| | | | decisively that it is an exceptional experience. Immediately |
| | | | after the use of your cream, there is a significant |
| | | | improvement in the appearance of the skin. The skin began |
| | | | to look fresh and glowing, and after a few days it seemed to |
| | | | be naturally tense and young. Beyond the delicate texture |
| | | | and the pleasant scent for the first time, I found results that |
| | | | surprised and astonished me. To be honest, I began with |
| | | | skepticism as I've been fed up with past false promises. |
| | | | Today, I receive many compliments on a daily basis about |
| | | | the appearance my skin from everybody, and especially |
| | | | have a great pleasure in looking at the mirror. |
| | | | A month later: |
| | | | And with the food comes the appetite . . . Recently, I began to |
| | | | apply the cream to the décolletage. Needless to say, as a |
| | | | mature woman, the force of gravity did not skip it and the |
| | | | signs were shown, which created frustration, discomfort and |
| | | | lack of self-confidence. At the same time, I was never |
| | | | interested in a plastic surgery. After two weeks of use I was |
| | | | amazed and happy to realize that the loose and soggy skin |
| | | | appeared tense and firm, fresh and glowing, and the breasts |
| | | | are lifted and lifted . . . , a sight I have not seen for many years. |
| | | | I have never experienced such an effect with any skin care |
| | | | product. |
| 12 | M. D. | After a month of | I am 52 years old M. D. Skin care is very important to me |
| | | experience with the | and for many years I have been using leading cosmetic |
| | | cream, the small | brands. After a month of experience with your cream, the |
| | | wrinkles | small wrinkles significantly reduced (small, because I do not |
| | | significantly reduced | have large). I enjoy the product very much and strongly |
| | | | recommend! |
| 13 | N. S. | After a few days of | After a few days of use of the cream I have already felt a |
| | | use of the cream I | change in facial appearance. The skin looked tighter and |
| | | have already felt a | brighter. Since then, the look has been improving wrinkles |
| | | change in facial | are fading up in the skin, spots become blurred, and the pores |
| | | appearance. The skin | are almost completely obscured. People pay attention and |
| | | looked tighter and | compliment me for my improved look. I am very very |
| | | brighter. | pleased. |

### SUMMARY

1. All subjects who gave their feedback noted that Composition #1 met or exceeded their expectations from a daily cream. They noted high efficacy in treating symptoms of ageing skin.

2. Specifically, the composition dramatically reduced the appearance of wrinkles.

3. The addition of nicotinamide solved the previous issues (Study #1) of burning sensation and over-tingling.

### Example 4: Gel Formulations Comprising Arginine or Citrulline and Nicotinamide

| Ingredients | #3 | #4 | #5 | #6 | #7 | #8 |
|---|---|---|---|---|---|---|
| Arginine | 8% | 10% | — | 10% | 10% | |
| Citrulline | | | 12% | | | 12% |
| Nicotinamide | 2% | 2% | 2% | 4% | 2% | 2% |
| Carbopol 940 | 0.4% | 0.5% | 0.6% | — | — | — |
| Methyl | — | — | — | 1% | 3% | 6% |
| cellulose | | | | | | |
| Propylene | 10% | 10% | 10% | 10% | 10% | 10% |
| glycol | | | | | | |
| Triethanol | 0.5% | 0.5% | 0.5% | 0.5% | 0.5% | 0.5% |
| amine | | | | | | |
| Preservative | 0.3% | 0.3% | 0.3% | 0.3% | 0.3% | 0.3% |
| Purified water | 100% | 100% | 100% | 100% | 100% | 100% |
| upto | | | | | | |

### Example 5: Serum Formulations Comprising Arginine or Citrulline and Nicotinamide

| Ingredients | #9 | #10 | #11 | #12 | #13 |
|---|---|---|---|---|---|
| Arginine | 8% | 10% | — | 10% | |
| Citrulline | | | 12% | | 10% |
| Nicotinamide | 2% | 2% | 2% | 4% | 2% |
| Hyaluronic acid HMW | 0.2% | 0.2% | 0.2% | 0.2% | 0.2% |
| Glycerine | 5% | 5% | 5% | 5% | 5% |
| PEG 12 dimethicon | 3% | 3% | 3% | 3% | 3% |
| Sodium diacrylate | 1% | 1% | 1% | 1% | 1% |
| Phenoxyethanol | 1% | 1% | 1% | 1% | 1% |
| Purified water upto | 100% | 100% | 100% | 100% | 100% |

### Example 6: A Day Cream Formulation

| | | Conc. |
|---|---|---|
| | | % |
| Ingredient Name | INCI Designation | W/W |
| Water | Water (aqua) | QS |
| Citric acid 50% soln | Water and citric acid | 10.94 |
| L-arginine c grade | Arginine | 10.00 |
| Stearin triple pression | Stearic acid | 8.00 |
| Amino acid Complex | Water (and) butylene glycol (and) lysine (and) histidine | 5.00 |
| | (and) arginine (and) aspartic acid (and) threonine (and) | |
| | serine (and) glutamic acid (and) proline (and) alanine | |
| | (and) valine (and) isoleucine (and) leucine (and) tyrosine | |
| | (and) phenylalanine | |
| Gransil GCM-5 | Cyclopentasiloxane and polysilicone-11 | 4.50 |
| Xiameter PMX-0245 | Cyclopentasiloxane | 3.50 |
| MPdiol | Methylpropanediol | 3.00 |
| Jeechem OP | Ethylhexyl palmitate | 2.50 |
| Cerasynt IP | Glycol stearate (and) stearamide amp | 2.00 |
| Essential water blend | Chamomilla recutita (matricaria) flower water | 2.00 |
| n21561 (mod of h5464) | zingiber officinale (ginger) water | |
| Unimoist U-125 G | Glycerin (and) Urea (and) Saccharide Hydrolysate (and) | 2.00 |
| | Magnesium Aspartate (and) Glycine (and) Alanine (and) | |
| | Creatine | |
| Sodium hydroxide 20% | Water and sodium hydroxide | 1.41 |
| soln. | | |
| Mikrokill COS | Phenoxyethanol (and) chlorphenesin (and) capryloyl | 1.25 |
| | glycol | |
| Hydreis | Hydrolyzed Beta-Glucan | 1.00 |
| Lipowax D | Cetearyl alcohol and ceteareth-20 | 1.00 |
| Veegum R | Magnesium aluminum silicate, purified smectite clay | 1.00 |
| Glycerin 99.7% USP | Glycerin | 0.50 |
| Lipovol A (avocado oil) | Persa gratissima (avocado) oil | 0.50 |
| Pleiotanicals ® | Aqua, glycerin and camellia sinensis leaf extract | 0.50 |
| Scavenox ™ GTE | | |
| Keltrol CG-SFT | Xanthan gum | 0.35 |
| Primalhyal 3000 | Hyaluronic Acid | 0.30 |
| Niacinamide PC | Niacinamide | 0.25 |
| Fresh & clean #28353 | Fragrance | 0.20 |
| Ursolic acid sodium salt | Sodium ursolate (and) sodium oleanolate | 0.20 |
| Na₂EDTA | Disodium EDTA | 0.10 |
| Oristar GLC | Glycine | 0.10 |
| Tenox BHT | BHT | 0.05 |
| Vc-PMG - magnesium | Magnesium ascorbyl phosphate | 0.05 |
| ascorbyl phosphate | | |
| Vitamin E acetate | Tocopheryl acetate | 0.05 |

### Example 7: a combination, comprising an NO-donor, a counter-NO agent, and a neurotoxin-mimetic agent.

### 7A—a composition comprising arginine, nicotinamide and neurotoxin-mimetic agents.

To the formulations of Example 5, add 5% Argireline®, 2% Syn-Ake® and 3% MunapsysTM

### 7B—a kit comprising arginine, nicotinamide and neurotoxin-mimetic agents

The kit comprises two topical compositions:

The first composition has the formulations of Example 5.

The second composition is a topical product, containing an emulsion-based base cream, to which 5% Argireline®, 2% Syn-Ake®, and 3% Munapsys™ are added.

Each of the first composition and the second composition is filled into a discrete container, suitable for administering such a composition onto the skin. The two compositions of the kit can be applied to the skin concurrently, or serially, for example, the first composition in the morning and the second composition in the evening.

What is claimed is:

1. A topical composition for application to the skin, comprising an NO-donor, and a counter-NO agent which balances the effect of the NO-donor in the skin, and cosmetically or pharmaceutically acceptable salts, isomers, analogs, or derivatives thereof.

2. The composition of claim 1, wherein the NO-donor is selected from the group consisting of an inorganic nitrite, a nitrate salt, an organic nitrite, an organic nitrate, an ester of nitric acid, sodium nitroprusside, molsidomine, diazeniumdiolates, S-nitrosothiols, mesoionic oxatriazole, an iron-sulphur nitrosyl, a nitrite ester of a sugar, a nitrate ester of a sugar, a nitrite ester of a polyol, a nitrate ester of a polyol, arginine, citrulline, nitroglycerin, ethylene glycol dinitrate, isopropyl nitrate, amyl nitrite, amyl nitrate, ethyl nitrite, butyl nitrite, isobutyl nitrite, octyl nitrite, glyceryl-1-mononitrate, glyceryl-1,2-dinitrate, glyceryl-1,3-dinitrate, butane-1,2,4-triol-trinitrate, erythrityl tetranitrate, pentaerythrityl tetranitrate, sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate, propatylnitrate, nicorandil, apresoline, diazoxide, hydralazine, hydrochlorothiazide, minoxidil, pentaerythritol, tolazoline; and cosmetically or pharmaceutically acceptable salts, isomers, analogs and derivatives thereof

3. The composition of claim 1, wherein the NO-donor is L-arginine, a salt, an isomer, an analog, or a derivative thereof, and wherein the concentration of L-arginine is selected from the group consisting of about 6% to about 25%, about 6% to about 12%, about 6% to about 8%, about 8% to about 10%, about 10% to about 14%, about 14% to about 18%, and about 18% to about 25%.

4. The composition of claim 1, wherein the counter-NO agent is nicotinamide, a salt, an isomer, an analog, or a derivative thereof.

5. The composition of claim 4, wherein the concentration of nicotinamide is selected from the group consisting of about 0.25% to about 10%, about 0.25% to about 4%, about 1% to about 2%, about 2% to about 4%, about 4% to about 6%, about 6% to about 8%, and about 8% to about 10%.

6. The composition of claim 1, wherein the ratio between the NO-donor and the counter-NO agent in the topical composition is selected from the group consisting of about 3:5 to about 100:1, about 2.5:1 to about 40:1, about 1:2 to about 8:1, about 1:2 to about 1:1, about 1:1 to about 2:1, about 2:1 to about 4:1, and about 4:1 to about 8:1.

7. The composition of claim 1, wherein the NO-donor is arginine and the counter-NO agent is nicotinamide, and wherein the ratio between the arginine and nicotinamide in the topical composition is selected from the group consisting of about 2.5:1 to about 40:1, about 3:5 to about 100:1, about 2.5:1 to about 5:1, about 2:1 to about 8:1, about 2:1 to about 4:1, about 4:1 to about 6:1, and about 6:1 to about 8:1.

8. The composition of claim 1, further comprising at least one neurotoxin.

9. The composition of claim 8, wherein the neurotoxin is selected from the group consisting of botulism toxin, an inhibitor of SNARE complex formation and catecholamine release, an antagonism of muscular nicotinic acetylcholine receptor, an inhibitor of the release of the acetylcholine (ACh) neurotransmitter into the synapse, acetyl hexapeptide-3, the peptide AC-gly glu-met-gln-arg-arg-NH2, Tripeptide-3, beta-Ala-Pro-Dab-NH-benzyl x2AcOH, acetyl hexapeptide-1, an antagonist at the acetylcholine postsynaptic membrane receptor, Pentapeptide-3, Lys-Thr-Thr-Lys-Ser; and derivatives, analogs or salts thereof.

10. The composition of claim 1, further comprising one or more inhibitors of hyaluronidase.

11. The composition of claim 1, wherein the composition is in a form selected from the group consisting of a liquid, a solution, an emulsion, a lotion, a cream, a gel, a foam, a stick, a lipstick, a mask, and a serum.

12. A kit comprising a first topical composition for application to the skin according to claim 1, and a second composition comprising at least one neurotoxin.

13. The kit of claim 12, wherein the at least one neurotoxin is selected from the group consisting of acetyl hexapeptide-3, beta-Ala-Pro-Dab-NH-benzyl×2AcOH, acetyl hexapeptide-1, Lys-Thr-Thr-Lys-Ser, botulism toxin, an inhibitor of SNARE complex formation and catecholamine release, an antagonism of muscular nicotinic acetylcholine receptor, an inhibitor of the release of the acetylcholine (ACh) neurotransmitter into the synapse, the peptide AC-gly glu-met-gln-arg-arg-NH2, Tripeptide-3, an antagonist at the acetylcholine postsynaptic membrane receptor, Pentapeptide-3, and derivatives, analogs, or salts thereof.

14. The kit of claim 12, wherein the second composition comprises about 5% acetyl hexapeptide-3, about 2% beta-Ala-Pro-Dab-NH-benzyl×2 AcOH, and about 3% acetyl hexapeptide-1.

15. The kit of claim 12, wherein the first composition or the second composition further comprise one or more inhibitors of hyaluronidase.

16. A method of treating, alleviating, or preventing a skin condition, comprising administering topically to the skin of a subject an effective amount of the composition of claim 1.

17. The method of claim 16, wherein the skin condition is associated with skin ageing, optionally wherein the skin is afflicted with at least one of the following: light-induced skin ageing, increased trans-epidermal water loss, skin dryness, fine lines, wrinkles, hyperpigmentation, and skin discoloration.

18. The method of claim 16, wherein said composition is a first composition and wherein said method further comprises applying to the skin a second composition comprising at least one neurotoxin, and wherein the application to the skin of the first composition is conducted in concurrence with, prior to, or following the administration of the second composition.

19. The method of claim 18, wherein the application of the composition augments the effect of the neurotoxin and/or prolongs the duration of the effect of the neurotoxin, and/or alleviates the inflammation and or the atrophy that is induced following administration of the neurotoxin.

20. The method of claim 18, wherein the first composition or the second composition further comprise one or more inhibitors of hyaluronidase.
